(19) United States
(12) Patent Application Publication
(10) Pub. No.: US 2006/0229364 A1
(43) Pub. Date: Oct. 12, 2006

(54) Antiviral compositions and methods of use

(75) Inventors: Terry R. Hobbs, St. Paul, MN (US); Steven S. Kantner, St. Paul, MN (US); Matthew T. Scholz, Woodbury, MN (US); Jeffrey F. Andrews, Stillwater, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/077,864

(22) Filed: Mar. 10, 2005

(51) Int. Cl. A61K 31/22

(52) U.S. Cl. 514/547

(57) ABSTRACT

Antiviral compositions, especially those useful when applied topically, particularly to mucosal tissues (i.e., mucous membranes), including, in particular, an antiviral lipid component, such as a fatty acid ester, fatty ether, or alkoxide derivative thereof. Such compositions provide effective topical antimicrobial activity and are accordingly useful in the treatment and/or prevention of conditions that are caused, or aggravated by, microorganisms (including viruses).

### BACKGROUND

The use of antimicrobial agents (e.g., antibiotics, antiseptics) plays an important part in current medical therapy. This is particularly true in the fields of dermatology as well as skin and wound antisepsis, where the most effective course of treatment for skin or mucous membranes, which are afflicted with bacterial, fungal, or viral infections or lesions, frequently includes the use of a topical antimicrobial agent.

Dermal afflictions caused by viral infections, such as cold sores and shingles, originate from inside the body. Infections caused by the herpes virus (e.g., herpes simplex virus 1 or 2, referred to as “HSV”), commonly known as “fever blisters” or “cold sores,” are common. Approximately 80% of American adults are infected with HSV-1, and an estimated 20-40% of adults suffer from recurrent outbreaks as described in Higgins CR, et al., Natural History, management and complications of herpes labialis, J. Med. Virol. 1 (Suppl.):22-26, 1993. Many known antiviral compounds may be unsuitable for topical treatment of these infections because they have limited ability to penetrate the skin.

Many topical compostions containing known antiviral compounds may fail to relieve the symptoms such as pain, inflammation and/or itchiness often associated with the dermal viral infection or skin lesion. Further, many may fail to prevent the secondary infection of these lesions by bacteria or fungi, leading to prolonged disease states and the potential for permanent scarring.

Thus, there is still a need for additional antiviral compositions.

### SUMMARY OF THE INVENTION

The present invention provides antiviral compositions and methods of using and making the compositions. Such compositions are typically useful when applied topically, particularly to skin, wounds, or mucosal tissues (i.e., mucous membranes), although a wide variety of surfaces can be treated. They can provide effective reduction, inhibition, prevention, or elimination of microbes, particularly viruses. The compositions also provide reduction or prevention of lesions caused by viruses, resulting in clinical improvement.

Compositions of the present invention provide effective topical antiviral activity and are accordingly useful in the local treatment and/or prevention of conditions that are caused, or aggravated by, viruses on various mammalian tissues, particularly skin, wounds, and/or mucous membranes.

Certain embodiments of the present invention also provide effective reduction, prevention, or elimination of other microbes including bacteria and fungi and hence can be can be particularly useful at treating secondary bacterial or fungal infections that often accompany the primary viral infection. Such compostions may include an enhancer component (i.e. an enhancer).

Significantly, certain embodiments of the present invention have a very low potential for generating microbial resistance. Thus, such compositions can be applied multiple times over one or more days to treat topical infections or to eradicate unwanted bacteria. Furthermore, compositions of the present invention can be used for multiple treatment regimens on the same patient without the fear of generating antimicrobial resistance.

Also, preferred compositions of the present invention have a generally low irritation level for skin, skin lesions, and mucosal membranes.

Compositions of the present invention include an antiviral lipid component. In certain embodiments, the antiviral lipid component includes a fatty acid ester of a polyhydric alcohol, a fatty ether of a polyhydric alcohol, a fatty alcohol ester of a hydroxyacid, alkoxylated derivatives thereof (of either the fatty acid ester, ether, or fatty alcohol ester), or combinations thereof. Certain of these antiviral lipids appear to have the ability to migrate through the stratum corneum, providing antiviral activity deeper into the skin that just at the surface.

Certain compositions further include an external analgesic component to provide relief to symptoms, such as pain and/or itch relief. Surprisingly, the ability of certain antiviral lipid components to permeate the skin appears to enhance the effectiveness of the external analgesic. Other components that can be included as well are thickeners, moisturizers including emollients and humectants, skin protectants, flavorants, other cosmetic or pharmaceutical actives, and surfactants.

Importantly, compositions of the present invention are capable of destroying microorganisms on or in mammalian tissue. Therefore, concentrations of components employed are generally greater than those that have been used to simply preserve certain topically applied compositions, i.e., prevent the growth of microorganism in topical compositions for purposes other than antisepsis.

In one embodiment, the present invention provides an antiviral composition that includes: an effective amount of an antiviral lipid component that includes a (C7-C14) saturated fatty acid monoester of a polyhydric alcohol, a (C8-C22) unsaturated fatty acid monoester of a polyhydric alcohol, a (C7-C14) saturated fatty monoether of a polyhydric alcohol, a (C8-C22) unsaturated fatty monoether of a polyhydric alcohol, a (C7-C14) saturated fatty alcohol ester of a (C2-C8) hydroxyacid, a (C8-C22) mono- or poly-unsaturated fatty alcohol ester of a (C2-C8) hydroxyacid, an alkoxylated derivative of any of the above wherein the alkoxylated derivative has less than 5 moles of alkoxide per mole of polyhydric alcohol, or combinations thereof, and an externa analgesic.

Preferably, the antiviral lipid component is present in an amount of greater than 5 wt-%, more preferably greater than 10 wt-%, even more preferably greater than 15 wt-%, and even more preferably greater than 20 wt-%. Unless otherwise specified, all weight percents are based on the total weight of a “ready to use” or “as used” composition. Preferably, if the antiviral lipid component includes a monoester of a polyhydric alcohol, a monoether of a polyhydric alcohol, or an alkoxylated derivative thereof, then there is no more than 50 wt-%, more preferably no more than 40 wt-%, even more preferably no more than 25 wt-%, and even more preferably no more than 15 wt-% of a diester, diether, triester, triether, or alkoxylated derivative thereof present, based on the total weight of the antiviral lipid component.

Preferably, the antiviral lipid component includes a (C8-C12) fatty acid ester of propylene glycol. In most embodiments the antiviral lipid component comprises propylene glycol monolaurate, propylene glycol monocaprate, propylene glycol monocaprylate, and combinations thereof.

Preferably, the antiviral composition includes an external analgesic. Safe and effective external analgesics include those selected from the amine and “caine” type, those selected from the alcohols and ketones type, those selected from the antihistamine type, those selected from hydrocortisone preparations, and mixtures thereof. When used in an appropriate wt-%, they temporary relieve the symptoms, such as pain or itch, associated with the viral infection. Preferred amine and “caine” type external analgesics include benzocaine, butamben picrate, dibucaine (or dibucaine HCl), dimethisoquin HCl, dyclonine HCl, lidocaine (or lidocaine HCl), pramoxine HCl, tetracaine (or tetracaine HCl), and mixtures thereof. Preferred alcohol and ketone type external analgesics include benzyl alcohol, camphor, camphorated metacresol, juniper tar, menthol, phenol, phenolate sodium, resorcinol, and mixtures thereof. Preferred antihistamine type external analgesics include diphenhydramine HCl, tripelennamine HCl, and mixtures thereof. Preferred hydrocortisone preparations include hydrocortisone, hydrocortisone acetate, and mixtures thereof. Mixtures of external analgesics from more than one type are also useful. Further information concerning safe and effective analgesics is provided in the Tentative Final Monograph on External Analgesic Drug Products for Over-the-counter Human Use, published by the United States Food and Drug Administration in the Federal Register, Volume 48, Number 27, Feb. 8, 1983, pages 5852 to 5869.

Preferably, the antiviral composition includes a moisturizer. The moisturizer can be a hydrophilic component including humectants such as propylene glycol, dipropylene glycol, polyethylene glycols, glycerol, sorbitol, alpha-hydroxy acids, urea, amino acids, ethoxylated amides, sodium pyrrolidone carboxylic acid and combinations thereof. Additionally, the moisturizer can be a hydrophobic occlusive component which helps to retain moisture including emollients such as mineral oil, squalene, petrolatum, cocoa butter, beeswax, jojoba oil, lanolin and derivatives, silicones, fatty acids, fatty alcohols, fatty acid esters, fatty alcohol esters, fatty acid triglycerides, and combinations thereof.

Certain materials including some humectants or emollients are particularly useful at providing safe and effective skin protection. Preferred skin protectants include allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

The present invention also provides methods of use of compositions of the present invention. In one embodiment, the present invention provides a method of preventing and/or treating an viral infection caused, or aggravated by, a microorganism on mammalian tissue, particularly skin and/or a mucous membrane. The method includes contacting the mammalian tissue, particularly skin and/or mucous membrane, with an antiviral composition of the present invention.

In other embodiments, the present invention provides methods for killing or inactivating microorganisms. Herein, to “kill or inactivate” means to render the microorganism ineffective by killing them (e.g., bacteria and fungi) or otherwise rendering them inactive (e.g., viruses). The present invention provides methods for inactivating enveloped viruses including but not limited to the viruses of the herpes family, such as Herpes Simpex I, Herpes Simplex II, Herpes Simplex VI, herpes zoster; poxviruses; corona viruses; paramyxoviruses; and togaviruses.

In certain embodiments, the composition of the present invention provides methods for killing bacteria and/or preventing bacterial infection for such as Staphylococcus spp., Streptococcus spp., Escherichia spp., Enterococcus spp., Pseudamonas spp. bacteria and combinations thereof, and more particularly Staphylococcus aureus (including antibiotic resistant strains such as methicillin resistant Staphylococcus aureus), Staphylococcus epidermidis, Escherichia coli (E. coli), Pseudomonas aeruginosa (Pseudomonasae), Streptococcus pyogenes, and combinations thereof which often are on or in the skin or mucosal tissue of a subject. The method includes contacting the microorganism with an antiviral composition of the present invention in an amount effective to kill one or more microorganisms (e.g., bacteria and fungi) or inactivate one or more microorganisms (e.g., viruses, particularly herpes virus).

In one embodiment, a method of treating lesions caused by viral infections is also provided. The method includes contacting the affected area with an antiviral composition that includes: an effective amount of an antiviral lipid component that includes a (C7-C14) saturated fatty acid ester of a polyhydric alcohol, a (C8-C22) unsaturated fatty acid ester of a polyhydric alcohol, a (C7-C14) saturated fatty ether of a polyhydric alcohol, a (C8-C22) unsaturated fatty ether of a polyhydric alcohol, a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, wherein the alkoxylated derivative has less than 5 moles of alkoxide per mole of polyhydric alcohol; and an external analgesic.

For example, in one embodiment, the present invention provides a method of treating a viral infection on mammalian tissue (particularly, the skin, mucosal tissue, and/or in a wound) of a subject. The method includes contacting the affected area with an antiviral composition that includes: an effective amount of an antiviral lipid component that includes a (C8-C14) fatty alcohol ester of a (C2-C8) hydroxyacid, a (C8-C22) mono- or poly-unsaturated fatty alcohol ester of a (C2-C8) hydroxyacid, an alkoxylated derivative thereof, or combinations thereof, wherein the alkoxylated derivative has less than 5 moles of alkoxide per mole of polyhydric alcohol.

In another embodiment, the present invention provides a method of topically treating a viral infection in mammals caused by the herpes family of viruses. Viral infections caused by the herpes family of viruses include cold sores, shingles, chicken pox, and genital herpes. The method includes contacting the affected area with an antiviral composition that includes: an effective amount of an antiviral lipid component that includes a (C7-C14) saturated fatty acid ester of propylene glycol, a (C8-C22) unsaturated fatty acid ester of a propylene glycol, or combinations thereof in an amount greater than 20 wt %.

In yet another embodiment, the present invention provides a composition useful for the topical treatment of an HSV infection and a method of topically treating said infection by contacting the affected area with an antiviral composition that includes: an effective amount of an antiviral lipid component that includes a (C7-C14) saturated fatty acid ester of propylene glycol, a (C8-C22) unsaturated fatty acid ester of a propylene glycol, or combinations thereof; in combination with an external analgesic. Suitable external analgesics include benzocaine, butamben picrate, dibucaine, dibucaine HCl, dimethisoquin HCl, dyclonine HCl, lidocaine, lidocaine HCl, pramoxine HCl, tetracain, tetracaine HCl, benzyl alcohol, camphor, camphorated metacresol, juniper tar, menthol, phenol, phenolate sodium, resorcinol, diphenhydramine HCl, tripelennamine HCl, hydrocortisone, hydrocortisone acetate, and mixtures thereof.

The compositions of the present invention can also be used for providing residual antimicrobial efficacy on a surface that results from leaving a residue or imparting a condition to the surface (e.g., skin, mucosal tissue, and/or wound) that remains effective and provides significant antimicrobial activity. This in particular may reduce the infectiousness of exanthemas, skin rashes, and lesions caused by measles, cold sores, chickenpox, hand foot and mouth disease, rubella, and roseola, among others. Further, such compositions may be used to prevent secondary bacterial infections at a viral site.

Methods of manufacture are also provided.

### DEFINITIONS

The following terms are used herein according to the following definitions.

“External analgesic” means a topically applied compound that has an analgesic, anesthetic, or antipruritic effect by depressing cutaneous sensory receptors, or that has a topical counterirritant effect by stimulating cutaneous sensory receptors.

“Effective amount” means the amount of the antiviral lipid component and/or the enhancer component when in a composition, as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level.

It should be understood that (unless otherwise specified) the listed concentrations of all components are for “ready to use” or “as used” compositions. The compositions can be in a concentrated form. That is, certain embodiments of the compositions can be in the form of concentrates that would be diluted by the user with an appropriate vehicle.

“Moisturizer” refers to a material that will increase the level of hydration of skin, mucous membrane, wound, lesion, or scab.

A “humectant” is a polar hygroscopic material that increases hydration by drawing water from the environment to help retain water in the skin's upper layers.

An “emollient” is a hydrophobic material that provides softness, lubricity, and smoothness to the skin and often forms a thin occlusive film which increases hydration by reducing transepidermal water loss (TEWL).

“Stable” means physically stable or chemically stable, which are both defined in greater detail below.

“Enhancer” means a component that enhances the effectiveness of the antimicrobial lipid component such that when the composition less the antiviral lipid component and the composition less the enhancer component are used separately, they do not provide the same level of antimicrobial activity as the composition as a whole. For example, an enhancer component in the absence of the antiviral lipid component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antiviral lipid component.

“Microorganism” or “microbe” or “microorganism” refers to bacteria, yeast, mold, fungi, protozoa, mycoplasma, as well as viruses (including lipid enveloped RNA and DNA viruses).

“Antibiotic” means an organic chemical produced by microorganisms that has the ability in dilute concentrations to destroy or inhibit microorganisms and is used to treat infectious disease. This may also encompass semi-synthetic compounds that are chemical derivatives of the compound produced by microorganisms or synthetic compounds that act on very specific biochemical pathways necessary for the cell's survival.

“Antiseptic” means a chemical agent that kills pathogenic and non-pathogenic microorganisms. Antiseptics generally interfere more broadly with the cellular metabolism and/or the cell envelope.

“Mucous membranes,” “mucosal membranes,” and “mucosal tissue” are used interchangeably and refer to the surfaces of the nasal (including anterior nares, nasoparangyl cavity, etc.), oral (e.g., mouth including the inner lip, buccal cavity and gums), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

“Antiviral lipid” means an antiseptic having at least one alkyl or alkylene group having at least 6 carbon atoms, preferably at least 7 carbon atoms, even more preferably at least 8 carbon atoms, and has a hydrophile/lipophile balance (HLB) of at most 6.2, more preferably at most 5.8, and even more preferably at most 5.5. The antiviral lipid preferably has an HLB of at least 3, preferably at least 3.2, and even more preferably at least 3.4.

“Fatty” as used herein refers to a straight or branched chain alkyl or alkylene moiety having at least 6 carbon atoms, unless otherwise specified.

“Affliction” means a condition to a body resulting from sickness, disease, injury, bacterial colonization, etc.

“Treat” or “treatment” means to improve the condition of a subject relative to the affliction, typically in terms of clinical symptoms of the condition.

“Subject” and “patient” includes humans, sheep, horses, cattle, pigs, dogs, cats, rats, mice, or other mammals.

“Wound” refers to an injury to a subject which involves a break in the normal skin or mucosal tissue barrier exposing tissue below, which is caused by, for example, lacerations, surgery, burns, damage to underlying tissue such as pressure sores, poor circulation, and the like. Wounds are understood to include both acute and chronic wounds.

“Lesion” as used herein is an abnormal condition of a tissue (e.g., skin and/or mucuous membrane) caused by a microbial (e.g., bacteria, viral, and/or fungal) infection.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an affliction means preventing, treating, or both treating and preventing further afflications).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides antimicrobial (including, e.g., antiviral, antibacterial, and antifungal) compositions. These compositions include one or more antiviral lipids, such as, for example, a fatty acid ester of a polyhydric alcohol, a fatty ether of a polyhydric alcohol, a fatty alcohol ester of a hydroxyacid, or alkoxylated derivatives thereof (of either the ester or ether). Certain compositions also include one or more external analgesics, and/or one or more moisturizers. In certain embodiments, the moisturizer can be the same as the antiviral lipid component.

The compositions of the present invention are useful for treating an infection caused by a herpes virus. The compositions, which include topical creams and ointments, are useful for treating topical skin infections caused by a herpes virus including but not limited to cold sores, shingles, and genital herpes. The formulations of this invention are useful for treating and preventing infections caused by a member of the herpes virus family.

The invention is particularly useful for treating and preventing cold sores caused by the herpes simplex I virus. About 15-20% of the adult population in the United States suffers occasionally from painful open lesions on the lips caused by this virus. The compositions are also useful for treating shingles, which is a painful rash of small blisters on a strip of skin anywhere on the body, most often on the trunk and buttocks. Shingles is caused by a herpes zoster virus. Animal models show that the formulations of this invention perform equally as well as commercial antiviral prescription products, particularly 5% acyclovir ointment. The formulations have the advantage over current drugs because they attack the lipid membrane in an antiseptic fashion and have a lower probability for developing antiviral resistance. Furthermore, the compositions will prevent the formation of a secondary bacterial infection in an open lesion or infection site. Hence, patients suffering with viral infections may be able to avoid other prophylactic antimicrobial treatments, such as oral antibiotics.

Such compositions adhere well to bodily tissues (i.e., mammalian tissues such as skin, mucosal tissue, and wounds) and thus are very effective topically. Thus, the present invention provides a wide variety of uses of the compositions. Particularly preferred methods involve topical application, particularly to skin (e.g., skin lesions) and wounds. Herein, such tissues are preferred examples of mammalian tissues.

Compositions of the present invention can be used to provide effective topical antimicrobial activity and thereby treat and/or prevent a wide variety of afflications. For example, they can be used in the treatment and/or prevention of afflictions that are caused, or aggravated by, microorganisms (e.g., Gram positive bacteria, Gram negative bacteria, fungi, protozoa, mycoplasma, yeast, viruses, and even lipid-enveloped viruses) on skin and/or mucous membranes, such as those in the nose, outer ear, and middle ear, mouth, rectum, vagina, or other similar tissues. Particularly relevant organisms that cause or aggravate such afflications include viruses of the herpes family, such as Herpes Simpex I, Hepres Simplex II, Herpes Simplex VI, herpes zoster; poxvirus, corona virus, paramyxovirus, and togavirus.

Compositions of the present invention can be used for the prevention and/or treatment of one or more microorganism-caused infections or other afflictions. In particular, compositions of the present invention can be used for preventing and/or treating cold sores.

The developmental stages of recurrent outbreaks caused by HSV-1 and/or HSV-2 are well known. The first, or prodromal stage, is characterized by normal appearance of skin accompanied by a tingling, burning, painful, or itching sensation. Subsequent stages include the formation of maculopapular lesions that develop into small, tense vesicles or blisters. The vesicles eventually break or collapse, with or without the formation of ulcers. Eventually, the lesion forms a crust. Overall, the lesion may last from seven to ten days.

Preferred compositions of the present invention can be used to treat outbreaks of lesions caused by HSV-1 and/or HSV-2. Application of the compositions can be applied at any stage of the outbreak of lesions to reduce the number of lesions and/or shorten the length of time of the outbreak. Application of the compositions during the prodromal stage may prevent or minimize the length or severity of the outbreak of lesions. Furthermore, they reduce the viral laod at the infection site.

Preferred compositions of the present invention contain an effective amount of antiviral lipid component to rapidly kill or inactivate microorganisms on skin, skin lesions, and mucosal membranes. Preferred compositions inactivate virions preventing transmission of an infectious virion from one person to another.

Preferred compositions of the present invention have a generally low irritation level for skin, skin lesions, and mucosal membranes.

Preferred compositions of the present invention are substantive for relatively long periods of time to ensure adequate efficacy. For example, certain compositions of the present invention remain at the site of application with antimicrobial activity for at least 4 hours and more preferably at least 8 hours.

In certain embodiments, the compositions may optionally include a penetration agent. A penetration agent is a compound that enhances the antiseptic diffusion into or through the skin or mucosal tissue by increasing the permeability of the tissue to the antimicrobial component and pharmacologically active agent, if present, to increase the rate at which the drug diffuses into or through the tissue. Examples of penetration agents are described in U.S. patent application Ser. No. ______, Attorney Docket No. 60655, filed Mar. 10, 2005.

Preferred compositions of the present invention are physically stable. As defined herein “physically stable” compositions are those that do not significantly change due to substantial precipitation, crystallization, phase separation, and the like, from their original condition during storage at 23° C. for at least 3 months, and preferably for at least 6 months. Particularly preferred compositions are physically stable if a 10-milliliter (10-ml) sample of the composition when placed in a 15-ml conical-shaped graduated plastic centrifuge tube (Corning) and centrifuged at 3,000 revolutions per minute (rpm) for 10 minutes using a Labofuge B, model 2650 manufactured by Heraeus Sepatech GmbH, Osterode, West Germany (or similar centrifuge at 2275×g) has no visible phase separation in the bottom or top of the tube.

Preferred compositions of the present invention exhibit good chemical stability. This can be especially a concern with the antiviral fatty acid esters, which can often undergo transesterification, for example. Preferred compositions retain at least 85%, more preferably at least 90%, even more preferably at least 92%, and even more preferably at least 95%, of the antiviral lipid component after aging for 4 weeks at 40° C. (an average of three samples) beyond the initial 5-day equilibration period at 23° C. The most preferred compositions retain an average of at least 97% of the antiviral lipid component after aging for 4 weeks at 40° C. in a sealed container beyond the initial 5-day equilibration period at 23° C. The percent retention is understood to mean the weight percent of antiviral lipid component retained. This is determined by comparing the amount remaining in a sample aged (i.e., aged beyond the initial 5-day equilibration period) in a sealed container that does not cause degradation, to the actual measured level in an identically prepared sample (preferably from the same batch) and allowed to sit at 23° C. for five days. The level of antiviral lipid component is preferably determined using gas chromatography as described in the Aging Study Using Gas Chromatography test method method described in U.S. patent application Ser. No. 10/937,059, filed Sep. 8, 2004.

Generally, the compositions of this invention may be in one of the following forms:

A hydrophobic or hydrophilic ointment: The compositions are formulated with a hydrophobic base (e.g., petrolatum, thickened or gelled water insoluble oils, and the like) and optionally having a minor amount of a water soluble phase. Hydrophilic ointments generally contain one or more surfactants or wetting agents.

An oil-in-water emulsion: The compositions may be formulations in which the antiviral lipid component is emulsified into an emulsion comprising a discrete phase of a hydrophobic component and a continuous aqueous phase that includes water and optionally one or more polar hydrophilic carrier(s) as well as salts, surfactants, emulsifiers, and other components. These emulsions may include water-soluble or water-swellable polymers as well as one or more emulsifier(s) that help to stabilize the emulsion. These emulsions generally have higher conductivity values, as described in U.S. patent application Ser. No. 09/966,511, filed on Sep. 28, 2001.

A water-in-oil emulsion: The compositions may be formulations in which the antiviral lipid component is incorporated into an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) that help to stabilize the emulsion.

Thickened Aqueous gels: These systems include an aqueous phase which has been thickened by suitable natural, modified natural, or synthetic polymers as described below. Alternatively, the thickened aqueous gels can be thickened using suitable polyethoxylated alkyl chain surfactants that effectively thicken the composition as well as other nonionic, cationic, or anionic emulsifier systems. Preferably, cationic or anionic emulsifier systems are chosen since some polyethoxylated emulsifiers can inactivate the antiviral lipids especially at higher concentrations.

Hydrophilic gels: These are systems in which the continuous phase includes at least one water soluble or water dispersible hydrophilic component other than water. The formulations may optionally also contain water up to 20% by weight. Higher levels may be suitable in some compositions. Suitable hydrophilic components include one or more glycols such as polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like. One skilled in the art will recognize that the level of ethoxylation should be sufficient to render the hydrophilic component water soluble or water dispersible at 23° C. In most embodiments, the water content is less than 20%, preferably less than 10%, and more preferably less than 5% by weight of the composition.

Antiviral Lipid Component

The antiviral lipid component is that component of the composition that provides at least part of the antiviral activity. That is, the antiviral lipid component has at least some antiviral activity for at least one virus. It is generally considered the main active component of the compositions of the present invention.

The antiviral lipids preferably have a hydrophile/lipophile balance (HLB) of at most 7.5, more preferably at most 5.8, and even more preferably at most 5.5. The antiviral lipids preferably have an HLB of at least 3, preferably at least 3.2, and even more preferably at least 3.4.

Preferred antiviral lipids are uncharged and have an alkyl or ankenyl hydrocarbon chain containing at least 7 carbon atoms.

In certain embodiments, the antiviral lipid component preferably includes one or more fatty acid esters of a polyhydric alcohol, fatty ethers of a polyhydric alcohol, fatty alcohol esters of a hydroxyacid, or alkoxylated derivatives thereof (of either or both of the esters and ether), or combinations thereof. More specifically and preferably, the antiviral lipid component is selected from the group consisting of a (C7-C14) saturated fatty acid ester of a polyhydric alcohol (preferably, a (C8-C12) saturated fatty acid ester of a polyhydric alcohol); a (C8-C22) unsaturated fatty acid ester of a polyhydric alcohol (preferably, a (C12-C22) unsaturated fatty acid ester of a polyhydric alcohol); a (C7-C14) saturated fatty ether of a polyhydric alcohol (preferably, a (C8-C12) saturated fatty ether of a polyhydric alcohol); a (C8-C22) unsaturated fatty ether of a polyhydric alcohol (preferably, a (C12-C22) unsaturated fatty ether of a polyhydric alcohol); a (C7-C14) saturated fatty alcohol monoester of a (C2-C8) hydroxycarboxylic acid (preferably, a (C7-C12) saturated fatty alcohol monoester of a (C2-C8) hydroxycarboxylic acid, more preferably, a (C8-C12) saturated fatty alcohol monoester of a (C2-C8) hydroxycarboxylic acid); a (C8-C22) mono- or poly-unsaturated fatty alcohol monoester of a (C2-C8) hydroxycarboxylic acid; an alkoxylated derivative of any of the foregoing; and combinations thereof. Various combinations of monoesters, diesters, monoethers, and diethers can be used in a composition of the present invention.

A fatty acid ester of a polyhydric alcohol is preferably of the formula R¹—C(O)—O—R², wherein R¹ is the residue of a (C7-C14) saturated fatty acid (preferably, a (C8-C12) saturated fatty acid), or a (C8-C22) unsaturated (preferably, a C12-C22) unsaturated, including polyunsaturated) fatty acid and R² is the residue of a polyhydric alcohol (typically and preferably, propylene glycol, although a wide variety of others can be used including pentaerythritol, sorbitol, ethylene glycol, hexylene glycol, polyglycerols, etc.). The R² group includes at least one free hydroxyl group (preferably, residues of glycerin, propylene glycol, or sucrose). Preferred fatty acid esters of polyhydric alcohols are esters derived from C8, C9, C10, C11, and C12 saturated fatty acids.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. As is generally know, 18:1, for example, means the compound has 18 carbon atoms and 1 carbon-carbon double bond. Preferred unsaturated chains have at least one unsaturated group in the cis isomer form.

In certain preferred embodiments, the fatty acid monoesters that are suitable for use in the present composition include known monoesters of propylene glycol monolaurate, propylene glycol monocaprate, propylene glycol monocaprylate, and combinations thereof. Propylene glycol monoesters are preferred because of their hydrolytic stability, liquid form, and ability to permeate the skin.

A fatty ether of a polyhydric alcohol is preferably of the formula R³—O—R⁴, wherein R³ is a (C7-C14) saturated aliphatic group (preferably, a (C8-C12) saturated aliphatic group), or a (C8-C22) unsaturated (preferably, (C12-C22) unsaturated, including polyunsaturated) aliphatic group and R⁴ is the residue of glycerin, sucrose, or propylene glycol. Preferred fatty ethers are monoethers of (C7-C14) alkyl groups (more preferably, (C8-C12) alkyl groups).

Exemplary fatty monoethers include, but are not limited to, laurylglyceryl ether, caprylglycerylether, caprylylglyceryl ether, laurylpropyleneglycol ether, caprylpropyleneglycol ether, and caprylylpropyleneglycol ether. Other fatty monoethers include glycerin and propylene glycol monoethers of oleyl (18:1), linoleyl (18:2), linolenyl (18:3), and arachonyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

A fatty alcohol ester of a hydroxyl functional carboxylic acid preferably has the formula:
R¹—O—(—C(O)—R²—O)ₙH
wherein R¹ is the residue of a (C7-C14) saturated alkyl alcohol (preferably, a (C7-C12) saturated alkyl alcohol, more preferably, a (C8-C12) saturated alkyl alcohol) or a (C8-C22) unsaturated alcohol (including polyunsaturated alcohol), R² is the residue of a hydroxycarboxylic acid wherein the hydroxycarboxylic acid has the following formula:
R³(CR⁴OH)ₚ(CH₂)_{q}COOH
wherein: R³ and R⁴ are each independently H or a (C1-C8) saturated straight, branched, or cyclic alkyl group, a (C6-C12) aryl group, or a (C6-C12) aralkyl or alkaryl group wherein the alkyl groups are saturated straight, branched, or cyclic, wherein R³ and R⁴ may be optionally substituted with one or more carboxylic acid groups; p=1 or 2; and q=0-3; and n=1, 2, or 3. The R³ group may include one or more free hydroxyl groups but preferably is free of hydroxyl groups. Preferred fatty alcohol esters of hydroxycarboxylic acids are esters derived from branched or straight chain C8, C9, C10, C11, and C12 alkyl alcohols. The hydroxyacids typically have one hydroxyl group and one carboxylic acid group. The hydroxycarboxylic acid moiety can include aliphatic and/or aromatic groups. For example, fatty alcohol esters of salicylic acid are possible. As used herein, a “fatty alcohol” is an alkyl or alkylene monofunctional alcohol having an even or odd number of carbon atoms.

Exemplary fatty alcohol monoesters of hydroxycarboxylic acids include, but are not limited to, (C8-C12) fatty alcohol esters of lactic acid such as octyl lactate, 2-ethylhexyl lactate (Purasolv EHL from Purac, Lincolnshire Ill., lauryl lactate (Chrystaphyl 98 from Chemic Laboratories, Canton Mass.), lauryl lactyl lacate, 2-ethylhexyl lactyl lactate; (C8-C12) fatty alcohol esters of 3-hydroxybutanoic acid, mandelic acid, gluconic acid, tartaric acid, and salicylic acid. Preferred fatty alcohol esters are C12 (or lauryl) alcohol esters.

The alkoxylated derivatives of the aforementioned fatty acid esters, fatty alcohol esters, and fatty ethers (e.g., one which is ethoxylated and/or propoxylated on the remaining alcohol group(s)) also have antimicrobial activity as long as the total alkoxylate is kept relatively low. In the case where the esters and ethers are ethoxylated, the total moles of ethylene oxide is preferably less than 5, and more preferably less than 2.

The fatty acid esters or fatty ethers of polyhydric alcohols or fatty alcohol esters of hydroxyacids can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The compositions of the present invention include one or more fatty acid esters, fatty alcohol esters, fatty ethers, alkoxylated fatty acid esters, alkoxylated fatty alcohol esters, or alkoxylated fatty ethers at a suitable level to produce the desired result. Such compositions preferably include a total amount of such material of greater than 5 percent by weight (wt-%), more preferably greater than 10 wt-%, even more preferably greater than 15 wt-%, even more preferably greater than 20 wt-%, and even more preferably at least 25 wt-%, based on the total weight of the “ready to use” or “as used” composition. In a preferred embodiment, they are present in a total amount of no greater than 95 wt-%, more preferably no greater than 90 wt-%, even more preferably no greater than 80 wt-%, and even more preferably no greater than 70 wt-%, based on the “ready to use” or “as used” composition. Certain compositions may be higher in concentration if they are intended to be diluted prior to use.

Preferred compositions of the present invention that include one or more fatty acid monoesters, fatty monoethers, or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antiviral lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antiviral lipid components present in the composition. However, as will be explained in greater detail below, higher concentrations of di- and tri-esters may be tolerated in the raw material if the formulation initially includes free glycerin because of transesterification reactions.

Although in some situations it is desirable to avoid di- or tri-esters as a component of the starting materials, it is possible to use relatively pure tri-esters in the preparation of certain compositions of the present invention (for example, as a hydrophobic component) and have effective antimicrobial activity.

External Analgesics

Safe and effective external analgesics include FDA-approved non-steroidal antiinflammatories, local anaesthetics, topical steroids and the like. Preferred analgesis include amines and “caine’ types; alcohols and ketones; antihistamines; hydrocortisone preparations; and mixtures thereof. Preferred amine and “caine” type external analgesics include benzocaine, butamben picrate, dibucaine (or dibucaine HCl), dimethisoquin HCl, dyclonine HCl, lidocaine (or lidocaine HCl), pramoxine HCl, tetracaine (or tetracaine HCl), and mixtures thereof prilocalne and mixtures thereof, such as EMLA (an eutectic mixture of local anaesthetic comprised of 2.5% lidocaine and 2.5% prilocalne). Preferred alcohol and ketone type external analgesics include benzyl alcohol, camphor, camphorated metacresol, juniper tar, menthol, phenol, phenolate sodium, resorcinol, and mixtures thereof. Preferred antihistamine type external analgesics include diphenhydramine HCl, tripelennamine HCl, and mixtures thereof. Preferred hydrocortisone preparations include hydrocortisone, hydrocortisone acetate, and mixtures thereof. Mixtures of external analgesics from more than one type are also useful.

When used in an appropriate wt-%, they temporary relieve the symptoms, such as pain, inflammation or itch associated with a viral infection. Preferred amounts of amine and “caine” type external analgesics include 5 to 20 wt-% benzocaine, 1 wt-% butamben picrate, 0.25 to 1 wt-% dibucaine (or dibucaine HCl), 0.3 to 0.5 wt-% dimethisoquin HCl, 0.5 to 1.0 wt-% dyclonine HCl, 0.5 to 5 wt-% lidocaine (or lidocaine HCl), 0.5 to 1 wt-% pramoxine HCl, 1 to 2 wt-% tetracaine (or tetracaine HCl), and mixtures thereof. Preferred amounts of alcohol and ketone type external analgesics include 10 to 33 wt-% benzyl alcohol, 0.1 to 3 wt-% camphor, camphorated metacresol (with 3 to 10.8 wt-% camphor and 1 to 3.6 wt-% metacresol), 1 to 5 wt-% juniper tar, 0.1 to 1 wt-% menthol, 0.5 to 1.5 wt-% phenol, 0.5 to 1.5 wt-% phenolate sodium, 0.5 to 3 wt-% resorcinol, and mixtures thereof. Preferred amounts of antihistamine type external analgesics include 1 to 2 wt-% diphenhydramine HCl, 0.5 to 2% tripelennamine HCl, and mixtures thereof. Preferred amounts of hydrocortisone preparations include 0.25 to 0.5 wt-% hydrocortisone, 0.25 to 0.5 wt-% hydrocortisone acetate, and mixtures thereof. Mixtures of external analgesics from more than one type are also useful.

For external analgesics, the Proposed Final Rulemaking for Fever Blister and Cold Sore Treatment Drug Products in the External Analgesic Drug Products for Over-the-counter Human Use Monograph, published by the United States Food and Drug Administration in the Federal Register, Volume 55, Number 21, 1/31/1990, pages 3370 to 3383 details: a) amine and “caine”-type local anesthetics including 1) 5 to 20% benzocaine, 7) 0.5 to 4% lidocaine, 9) 0.5 to 1% pramoxine hydrochloride, 10) 1 to 2% tetracaine, and b) alcohols and ketones including 1) 10 to 33% benzyl alcohol, 2) 0.1 to 3% camphor, 6) 0.1 to 1% menthol, 7) 0.5 to 1.5% phenol, 10) 0.5 to 3% resorcinol. Combinations of “a” with “b” are also permitted as are blends of menthol and/or camphor with benzyl alcohol, phenol, camphor, or other category b materials. A special combination of 3 to 10.8% camphor with 4.7% phenol combined in a light mineral oil is allowed.

Moisturizers

Compostions of the present invention may include a moisturizer to increase the level of hydration of skin, mucous membrane, wound, lesion, or scab. The moisturizer can be a hydrophilic material including humectants or it can be a hydrophobic material including emollients. A humectant is a polar hygroscopic material that increases hydration by drawing water from the environment to help retain water in the skin's upper layers. An emollient is a hydrophobic material that provides softness, lubricity, and smoothness to the skin and often forms a thin occlusive film that increases hydration by reducing transepidermal water loss (TEWL).

Hydrophilic moisturizers. Exemplary hydrophilic moisturizers include, but are not limited to, water, polyhydric alcohols, lower alkyl ethers, N-methylpyrrolidone, lower alkyl esters, urea, amino acids, ethoxylated amides, sodium pyrrolidone carboxylic acid, and the lower monohydroxy alcohols and hydroxy acids discussed below as enhancers, as well as combinations thereof. Thus, a lower monohydroxy alcohol can function as both a hydrophilic compound and an enhancer. Preferably, the hydrophilic components include polyhydric alcohols, lower alkyl ethers, and short chain esters. More preferably, the hydrophilic components include polyhydric alcohols.

Suitable polyhydric alcohols (i.e., organic compounds having more than one hydroxyl group) have a molecular weight of less than 500, preferably less than 400, and more preferably less than 200. Examples of polyhydric alcohols include, but are not limited to, glycerol, propylene glycol, dipropylene glycol, tripropylene glycol, polypropylene glycol, polyethylene glycol, diethylene glycol, pentaerythritol, trimethylolpropane, trimethylolethane, trimethylolbutane, sorbitol, mannitol, xylitol, pantothenol, ethylene glycol adducts of polyhydric alcohol, propylene oxide adducts of polyhydric alcohol, 1,3-butanediol, dipropylene glycol, diglycerine, polyglycerine, erythritol, sorbitan, sugars (e.g., sucrose, glucose, fructose, mannose, xylose, saccharose, trehalose), sugar alcohols, and the like. Certain preferred polyhydric alcohols include glycols (i.e., those containing two hydroxyl groups), glycerin and propylene glycol. Certain other preferred polyhydric alcohols include sucrose, xylitol, mannitol, and sorbitol.

Ethers include materials such as dimethylisosorbide, polyethylene glycol and methoxypolyethylene glycols, block and random copolymers of ethylene oxide and propylene oxide, and laureth-4. Alkyl esters include triacetin, methyl acetate, methyl lactate, ethyl lactate esters, esters of polyethoxylated glycols, and combinations thereof.

In certain preferred embodiments, the hydrophilic components useful in the compositions of the present invention include those selected from the group consisting of glycols, glycerin and propylene glycol, and mixtures thereof. Most preferably, the hydrophilic component is selected to match the polyhydric alcohol portion of any fatty acid monoester of a polyhydric alcohol antiviral present. For example, if the antiviral agent was glycerolmonolaurate (monolaurin) the most preferred hydrophilic component is glycerin. In this manner, any transesterification reaction that may occur with the carrier solvent does not produce an undesirable by-product. If there are other components in the composition that may esterify with hydroxylfunctional hydrophilic components, conditions are selected to minimize this occurrence. For example, the components are not heated together for extended periods of time, and/or the pH is close to neutral if possible, etc.

One or more hydrophilic materials may be used in the compositions of the present invention at a suitable level to produce the desired result. In certain preferred embodiments that also include the hydrophilic component as the primary component (i.e., the component used in the greatest amount and referred to as a “vehicle”), the hydrophilic component is present in a total amount of at least 0.1%, preferably at least 1 wt-%, more preferably at least 4 wt-%, and even more preferably at least 8 wt-%, based on the weight of the ready to use composition. In certain embodiments, higher levels of hydrophilic component may be employed. In these cases the hydrophilic component is present in a total amount of at least 10 wt-%, more preferably at least 20 wt-%, and even more preferably at least 25 wt-%.

In a preferred embodiment, the hydrophilic component is present in a total amount of no greater than 70 wt-%, preferably no greater than 60 wt-%, more preferably no greater than 40 wt-%, even more preferably no greater than 30 wt-%, based on the ready to use composition. When the hydrophilic component is present in the greatest amount it is referred to as a “vehicle.”

Hydrophobic Moisturizers. Exemplary hydrophobic moisturizers include, but are not limited to, short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH; (C2-C18) alkyl or (C6-C12) aryl esters of glycerol, pentaerythritol, ethylene glycol, propylene glycol, as well as polyethoxylated derivatives of these; (C12-C22) alkyl esters or (C12-C22) ethers of polypropylene glycol; (C12-C22) alkyl esters or (C12-C22) ethers of polypropylene glycol/polyethylene glycol copolymer; and polyether polysiloxane copolymers. Additional examples of hydrophobic components include cyclic dimethicones, including volatile cyclic silicones such as D4 and D5, polydialkylsiloxanes, polyaryl/alkylsiloxanes, silicone copolyols, cocoa butter, beeswax, jojoba oil, lanolin and derivatives, long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids; hydrocarbons including straight and branched chain alkanes and alkenes such as isoparafins (e.g., isooctane, isododecane, isooctadecane, etc.), squalene, and mineral oil, polysiloxane polyalkylene copolymers, dialkoxy dimethyl polysiloxanes; (C12-C22) alkyl and (C12-C22) alkenyl alcohols, and petroleum derived alkanes such as isoparafins, petrolatum, petrolatum USP, as well as refined natural oils (especially NF or USP grades) such as olive oil NF, cotton seed oil, castor oil, peanut oil, corn oil, seasame oil, safflower oil, soybean oil, and the like, and blends thereof. In certain preferred embodiments, the hydrophobic components useful in the compositions of the present invention include those selected from the group consisting of petrolatum USP and short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C 12) aryl esters of (C4-C 12) diacids or (C4-C 12) diols optionally substituted in available positions by —OH (such as diisopropyladipate, diisopropylsebacate); (C1-C9) alkyl or (C6-C12) aryl esters of glycerol, pentaerythritol, ethylene glycol, propylene glycol (such as glyceryl tricaprylate/caprate); and mixtures thereof.

Skin Protectants

Certain materials including some humectants or emollients are particularly useful at providing safe and effective skin protection. When used in appropriate wt-%, they temporarily protect injured or exposed skin or mucous membrane surfaces from harmful or annoying stimuli, and may help provide relief to such surfaces. Preferred skin protectants include 0.5 to 2 wt-% allantoin, 0.15 to 5 wt-% aluminum hydroxide gel, 1 to 25 wt-% calamine, 50 to 100 wt-% cocoa butter, 5 to 13.56 wt-% cod liver oil, at least 0.007 wt-% colloidal oatmeal, 1 to 30 wt-% dimethicone, 20 to 45 wt-% glycerin, 50 to 100 wt-% hard fat, 4 to 20 wt-% kaolin, 12.5 to 50 wt-% lanolin, 50 to 100 wt-% mineral oil, 30 to 100 wt-% petrolatum, sodium bicarbonate, 10 to 98 wt-% topical starch, 0.1 to 2 wt-% zinc acetate, 0.2 to 2 wt-% zinc carbonate, 1 to 25 wt-% zinc oxide, 0.13 to 0.5 wt-% aluminum acetate, 46 to 63 wt-% aluminum sulfate, and witch hazel. Further information concerning safe and effective skin protectants is provided in the Proposed Final Rulemaking for Fever Blister and Cold Sore Treatment Drug Products in the Skin Protectant Drug Products for Over-the-counter Human Use Monograph, published by the United States Food and Drug Administration in the Federal Register, Volume 51, Number 21, 1/31/1990, pages 3362 to 3370.

Enhancer Component

Compositions of the present invention may optionally include an enhancer (preferably a synergist) to enhance the antimicrobial activity especially against Gram negative bacteria, such as E. coli and Psuedomonas sp. The enhancer component may include an alpha-hydroxy acid, a beta-hydroxy acid, other carboxylic acids, a (C1-C4) alkyl carboxylic acid, a (C6-C12) aryl carboxylic acid, a (C6-C12) aralkyl carboxylic acid, a (C6-C12) alkaryl carboxylic acid, a phenolic compound (such as certain antioxidants and parabens), a (C1-C10) monohydroxy alcohol, a chelating agent, or a glycol ether (i.e., ether glycol) as described in U.S. patent application Ser. No. 10/937,059, filed Sep. 8, 2004. Various combinations of enhancers can be used if desired.

One or more enhancers may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition. Such concentrations typically apply to alpha-hydroxy acids, beta-hydroxy acids, other carboxylic acids, chelating agents, phenolics, ether glycols, (C5-C10) monohydroxy alcohols. Generally, higher concentrations are needed for (C1-C4) monohydroxy alcohols.

In a preferred embodiment, the short chain (i.e., C1-C4) alcohols are present in a total amount of at least 10 wt-%, even more preferably at least 15 wt-%, even more preferably at least 20 wt-%, and even more preferably at least 25 wt-%, based on the total weight of the ready to use composition.

In a preferred embodiment, the (C1-C4) alcohols are present in a total amount of no greater than 90 wt-%, more preferably no greater than 70 wt-%, even more preferably no greater than 60 wt-%, and even more preferably no greater than 50 wt-%, based on the total weight of the ready to use composition.

Surfactants

Compositions of the present invention optionally can include one or more surfactants to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. In preferred embodiments, the surfactant includes poloxomer, ethoxylated stearates, sorbitan oleates, high molecular weight crosslinked copolymers of acrylic acid and a hydrophobic comonomer, and cetyl and stearyl alcohols as cosurfactants.

A wide variety of conventional surfactants can be used; however, certain ethoxylated surfactants can reduce or eliminate the antimicrobial efficacy of the antiviral lipid component. The exact mechanism of this is not known and not all ethoxylated surfactants display this negative effect. For example, poloxamer (polyethylene oxide/polypropylene oxide) surfactants have been shown to be compatible with the antiviral lipid component, but ethoxylated sorbitan fatty acid esters such as those sold under the trade name TWEEN by ICI have not been compatible. It should be noted that these are broad generalizations and the activity could be formulation dependent. One skilled in the art can easily determine compatibility of a surfactant by making the formulation and testing for antimicrobial activity as described in U.S. patent application Ser. No. 10/937,059, filed Sep. 8, 2004. Combinations of various surfactants can be used if desired.

It should be noted that certain antiviral lipds are amphiphiles and may be surface active. For example, certain antiviral alkyl monoglycerides described herein are surface active. For certain embodiments of the invention, the antiviral lipid component is considered distinct from a “surfactant” component.

Thickeners

For certain applications, it may be desirable to formulate the antiviral lipid in compositions that are thickened with soluble, swellable, or insoluble organic polymeric thickeners such as natural and synthetic polymers including polyacrylic acids, poly(N-vinyl pyrrolidones), cellulosic derivatives, and xanthan or guar gums or inorganic thickeners such as silica, fumed silica, precipitated silica, silica aerogel and carbon black, and the like; other particle fillers such as calcium carbonate, magnesium carbonate, kaolin, talc, titanium dioxide, aluminum silicate, diatomaceous earth, ferric oxide and zinc oxide, clays, and the like; ceramic microspheres or glass microbubbles; ceramic microspheres suc as those available under the tradenames “ZEOSPHERES” or “Z-LIGHT” from 3M Company, St. Paul, Minn. The above fillers can be used alone or in combination.

Optional Additives

Compositions of the present invention may additionally employ adjunct components conventionally found in cosmetic and pharmaceutical compositions in their art-established fashion and at their art-established levels. Thus, for example, the compositions may contain additional compatible pharmaceutically active materials for combination therapy (such as supplementary antimicrobials, anti-parasitic agents, antipruritics, astringents, healing promoting agents, steroids, non-steroidal anti-imflammatory agents, or other anti-inflammatory agents), or may contain materials useful in physically formulating various dosage forms of the present invention, such as excipients, dyes, pigments, perfumes, fragrances, lubricants, thickening agents, stabilizers, skin penetration enhancers, preservatives, film forming polymers, or antioxidants. The compositions may also contain vitamins such as vitamin B, vitamin C, vitamin E, vitamin A, and derivates thereof.

Bioadhesive polymers optionally may be added. Numerous suitable bioadhesive polymers are discussed in International Publication No. WO 93/21906. Representative bioadhesive polymers of particular interest include bioerodible hydrogels described by H. S. Sawhney et al., in Macromolecules, 26:581-587 (1993), including polyhyaluronic acids, casein, gelatin, glutin, polyanhydrides, polyacrylic acid, alginate, chitosan, poly(methyl methacrylates), poly(ethyl methacrylates), poly butylmethacrylate), poly(isobutylmethacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Preferred polymers are polyacrylic acid (e.g., CARBOMER polymers) and poly(fumaric-co-sebacic)acid. Other bioadhesive and bioerodible polymers are described in U.S. Pat. No. 6,746,635. Particularly preferred are slightly crosslinked polyacrylic acids such as those sold under the CARBOPOL brand by BF Goodrich.

It will also be appreciated that additional antiseptics, disinfectants, antivirals, or antibiotics may be included and are contemplated. These include, for example, addition of metals such as silver, copper, zinc; iodine and iodophors; chlorhexidine and its various salts such as chlorhexidine digluconate; polyhexamethylenebiguanide, parachlorometaxylenol, triclosan, antimicrobial quaternary amines including benzethonium chloride, benzalkonium chloride, and polymeric quaternary amines, “azole” antifungal agents including clortrimazole, miconazole, econazole, ketoconazole, and salts thereof; and the like. Antibiotics such as neomycin sulfate, bacitracin, mupirocin, polymyxin, rifampin, tetracycline, and the like, also may be included. Preferred compositions, however, are free of antibiotics due to the chance of resistance formation. Antiviral agents incluce, but are not limited to: acydouir, valaciolair, Pencidouir, and famcidouir.

It will be appreciated by the skilled artisan that the levels or ranges selected for the required or optional components described herein will depend upon whether one is formulating a composition for direct use, or a concentrate for dilution prior to use, as well as the specific component selected, the ultimate end-use of the composition, and other factors well known to the skilled artisan.

Many of the compositions of the present invention have exceptional broad spectrum antimicrobial activity and thus are generally not terminally sterilized but if necessary may be sterilized by a variety of industry standard techniques. For example, it may be preferred to sterilize the compositions in their final packaged form using electron beam. It may also be possible to sterilize the sample by gamma radiation or heat. Other forms of sterilization may be acceptable. It may also be suitable to include preservatives in the formulation to prevent growth of certain organisms. Suitable preservatives include industry standard compounds such as Parabens (methyl, ethyl, propyl, isopropyl, isobutyl, etc), 2 bromo-2 nitro-1,3 diol; 5 bromo-5-nitro-1,3 dioxane, chlorbutanol, diazolidinyl urea; iodopropylnyl butylcarbamate, phenoxyethanol, halogenated cresols, methylchloroisothiazolinone and the like, as well as combinations of these compounds.

Formulations and Methods of Preparation

The compositions of the present invention preferably adhere well to mammalian tissues (particularly, skin, mucosal tissue, and wounds), in order to deliver the antiviral to the intended site over a prolonged period even in the presence of perspiration. The component in the greatest amount (i.e., the vehicle) in the formulations of the invention may be any conventional vehicle commonly used for topical treatment of human or animal skin. The hydrophobic ointment and the oil in water emulsion, which can take the form of a cream or lotion, are preferred embodiments of the present invention.

The formulations are typically selected from one of the following types:

(1) A hydrophobic ointment: The compositions are formulated with a hydrophobic base (e.g., petrolatum, thickened or gelled water insoluble oils, and the like) and optionally having a minor amount of a water soluble phase.

The hydrophobic ointment is an anhydrous or nearly anhydrous formulation with a hydrophobic vehicle. Typically the components of the ointment are chosen to provide a semi-solid consistency at room temperature which softens or melts at skin temperature to aid in spreading. Suitable components to accomplish this include low to moderate amounts of natural and synthetic waxes, for example beeswax, carnuba wax, candelilla wax, ceresine, ozokerite, microcrystalline waxes, and parafins. Viscous semi-crytalline materials such as petrolatum and lanolin are useful in higher amounts. The viscosity of the ointment can also be adjusted with oil phase thickeners including hydrophobically modified clays. In certain preferred embodiments of the present invention, the compositions are chosen to spread easily and absorb relatively rapidly into the epidermis. This rapid absorption is especially desirable when the composition is used to treat cold sores around the mouth as it limits the amount that is licked off or transferred to food. Rapid absorption is achieved by minimizing the amount of high melting waxes used and limiting the use of non-polar hydrocarbon materials such as petrolatum and mineral oil. Many of the prefered external analgesics and skin protectant materials described earlier are soluble in hydrophobic vehicles, particularly in the presence of the somewhat polar antiviral lipid component. For materials that aren't readily soluble, such as allantoin, or some of the enhancers, they can be suspended as solids in the ointment, or can be solubilized with a small amount of a hydrophilic component. For example, when formulating with organic acid enhancers or certain solid surfactants in petrolatum many enhancers and surfactants will dissolve into the petrolatum at temperatures above 85° C.; however, upon cooling, the enhancer and/or surfactant crystals or precipitates back out of solution making it difficult to produce a uniform formulation. If at least 0.1%, and preferably at least 1.0%, more preferably at least 2%, and most preferably at least 3 wt-%, of a hydrophilic compound (e.g., a glycol) is added, a stable formulation can be obtained. It is believed that these formulations produce an emulsion in which the enhancer and/or surfactant is dissolved, emulsified, or dispersed in the hydrophilic component which is emulsified into the hydrophobic component(s). These compositions are stable upon cooling and centrifuging.

Furthermore, it is believed that incorporation of the hydrophilic component in the formulation improves the antimicrobial activity. The mechanism for this is unknown; however, it may speed the release of the enhancer component and/or the antiviral lipid component.

The water content of these formulations is preferably less than 20%, preferably less than 10 wt-%, more preferably less than 5 wt-%, and even more preferably less than 2 wt-%, in order to minimize hydrolysis of any ester based antiviral lipid present.

Furthermore, it has been found that it is particularly desirable where the antiviral lipid component includes an ester to use either glycerin or propylene glycol in the hydrophilic component. It is most preferred to use a hydrophilic compound that is identical to the glycol portion of the antiviral lipid, e.g., propylene glycol with the propylene glycol esters and glycerin with the glycerin esters. In this manner, transesterification of the antiviral lipid ester with the hydrophilic compound will not result in additional chemical species present. In fact, there is some evidence to show that use of glycerolmonolaurate, which is 95% pure, when formulated with glycerin as a hydrophilic compound results in formation of additional glycerol monolaurate due to transesterification of the diester with the glycerin to produce two moles of the monoester. For this reason, it may be possible to initially formulate with lower grade glycerin ester that contains considerable levels of diester present, as long as it transesterifies during manufacture and/or storage to produce a formulation that includes less than 15% diester and preferably less than 5% diester based on the total weight of antiviral lipid present.

These formulations can be relatively easily manufactured by first heating the hydrophobic component to 85° C., adding in the skin protectant if different from the hydrophobic component, surfactant, hydrophilic component, and enhancer component, cooling to 65° C., and adding the external analgesic, antiviral lipid component above its melting point. Alternatively, the enhancer component can be predissolved in the hydrophilic component (optionally along with the surfactant) and added to the hydrophobic component either before or after addition of the antiviral lipid component. If either the antiviral lipid component or the hydrophobic component is a solid at room temperature this is done at the minimum temperature necessary to melt all components. Exposure of ester containing antiviral lipids to enhancers that include either acid or ether groups to elevated temperatures for extended periods of time should be avoided to prevent transesterification reactions (unless this is deliberate in the case of utilizing lower purity fatty acid esters in combination with glycol hydrophilic components to produce the monoesters as discussed above).

The viscosity of these formulations intended for use on skin is preferably at least 500 centipoise (cps), more preferably at least 1,000 cps, and even more preferably at least 10,000 cps. The viscosity can be measured by the Viscosity Test as described in U.S. patent application Ser. No. 10/937,059, filed Sep. 8, 2004.

Similarly the viscosity and/or melt temperature can be enhanced by either incorporating a crystalline or semicrystalline hydrophobic carrier such as a higher melting petrolatum, addition of an insoluble filler/thixotrope, or by addition of a polymeric thickener (e.g., a polyethylene wax in a petrolatum vehicle). Polymeric thickeners may be linear, branched, or slightly crosslinked. It is important for comfort that the formulations are relatively soft and that they spread easily to allow easy application, especially over a wound, rash, or infected area.

(2) An oil-in-water emulsion: The compositions may be formulations in which the antiviral lipid component is emulsified into an emulsion comprising a discrete phase of a hydrophobic component and a continuous aqueous phase that includes water and optionally one or more polar hydrophilic carrier(s) as well as salts, surfactants, emulsifiers, and other components. These emulsions may include water-soluble or water-swellable polymers as well as one or more emulsifier(s) that help to stabilize the emulsion. These emulsions generally have higher conductivity values, as described in U.S. patent application Ser. No. 09/966,511, filed on Sep. 28, 2001.

(3) A water-in-oil emulsion: The compositions may be formulations in which the antiviral lipid component is incorporated into an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) that help to stabilize the emulsion.

(4) Thickened aqueous gels: These systems include an aqueous phase which has been thickened by suitable natural, modified natural, or synthetic polymers. Alternatively, the thickened aqueous gels can be thickened using suitable polyethoxylated alkyl chain surfactants that effectively thicken the composition as well as other nonionic, cationic, or anionic emulsifier systems. Preferably, cationic or anionic emulsifier systems are chosen since some polyethoxylated emulsifiers can inactivate the antiviral lipids especially at higher concentrations.

(5) Hydrophilic gels: These are systems in which the continuous phase includes at least one water soluble hydrophilic component other than water. The formulations may optionally also contain water up to 20% by weight. Higher levels may be suitable in some compositions. Suitable hydrophilic components include one or more polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like.

(6) Oil in Water Emulsions. Antiviral lipid components of this invention can be formulated into oil-in-water emulsions in combination with external analgesics. Particularly preferred compositions comprise at least 35%, preferably at least 40%, more preferably at least 45%, and most preferably at least 50%, by weight water phase. As used herein the water phase includes all components which are soluble in water at 23° C. Several methods to produce stable oil-in-water emulsions are known to those skilled in the art including the use of stearate soaps, non-ionic surfactants, acrylates/C10-30 alkyl acrylate crosspolymers, and phase inversion emulsification. Generally speaking, the hydrophobic component (oil) is mixed in Container A along with any emulsifier(s) optionally including polymeric emulsifiers and heated to a temperature sufficient to ensure a homogenous composition and subsequent stable emulsion. For certain combinations of hydrophobic components, a homogeneous composition may result at room temperature and heating is not required. The temperature is typically raised to at least 60° C., preferably to at least 80° C., and more preferably to 100° C. or more. In a separate Container B, the hydrophilic ingredients are mixed, including one or more of the following: water, hydrophilic component, enhancer(s), surfactant(s), and acids/bases to adjust the pH of the final composition. The contents of container B are heated to a temperature sufficient to ensure a stable final emulsion composition without significantly degrading any of the components, typically to a temperature greater than 40° C., preferably greater than 50° C., and more preferably greater than 60° C. While hot, container B is added to container A using a high shear mixer. The composition may be continuously mixed until cool (e.g., to a temperature of less than 40° C.) or it can be allowed to sit as long as the contents remain uniformly mixed. If the antiviral lipid is heat sensitive, it is added with mixing during the cooling down period. If it is not heat sensitive, it may be added to either container A or container B. The viscosity of these compositions may be adjusted by altering the levels of emulsifier; changing the ratio of water to oil phase; selection of the oil phase (e.g., select from an oil (hydrophobic component), which is more or less viscous); incorporation of a polymeric or particulate thickener, etc.

(7) Neat Compositions. The compositions of the present invention also may be delivered to the treatment site in a neat form or in a volatile solvent that rapidly evaporates to leave behind a neat composition. This may be particularly suitable for delivery to the Eustachian tube but could also be utilized for delivery into the ear canal or to the surface of the tympanic membrane. Such compositions may be solid, semi-solid, or liquid. In the case where the compositions are solid, the antimicrobial and/or the enhancer and/or the surfactant may optionally be microencapsulated to either sustain the delivery or facilitate manufacturing a powder, which is easily delivered. Alternatively, the composition can be micronized into a fine powder without the addition of other components or it may optionally contain fillers and other ingredients that facilitate powder manufacture. Suitable powders include, but are not limited to, calcium carbonate, calcium phosphate, various sugars, starches, cellulose derivatives, gelatin, and polymers such as polyethylene glycols.

When hydrophobic antimicrobial lipids are used, a method for micronizing a hydrophobic agent may be used wherein the hydrophobic agent is dissolved in an effective amount of a first solvent that is free of polymer (such as the method described in U.S. Pat. No. 6,746,635). The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

Viscosity

Certain preferred compositions of the present invention have a viscosity of 500 Centipoise (cps) for ease of application topically. More preferably, compositions of the present invention have a viscosity of at least 1,000 cps, even more preferably at least 10,000 cps.

Delivery Methods and Devices

Topical treatment regimens according to the practice of this invention include applying a safe and effective amount of the compositions described herein directly to the infected or at-risk skin, wound, or mucous membrane. Typically, the compositions are delivered to the skin and/or mucosal tissue in a manner that allows them to penetrate into the skin and/or mucosal tissue, as opposed to through the tissue into the blood stream. This concentrates the compositions locally at the site in need of treatment. Preferably treatment is started at the prodromal stage of the viral infection, prior to the development of a rash, sore or exanthema. Delivery can be accomplished by spraying, dipping, wiping, dropping, pouring, toweling, or the like, onto the area to be treated.

In the methods of the present invention, the compositions may be provided as a formulation suitable for delivery to mammalian tissue (e.g., skin and/or mucosal surfaces). Suitable formulations can include, but are not limited to, creams, gels, foams, ointments, lotions, balms, waxes, salves, solutions, suspensions, dispersions, water in oil or oil in water emulsions, microemulsions, pastes, powders, oils, lozenges, boluses, and sprays, and the like.

Various other modes of administration can be used as well known to one of skill in the art depending on the desired location for contact of the antiviral compositions of the present invention.

For application to skin or mucosal tissue, for example, the compositions may be applied directly to the tissue from a collapsible container such as a flexible tube, blow/fill/seal container, pouch, capsule, etc. In this embodiment, the primary container itself is used to dispense the composition directly onto the tissue or it can be used to dispense the composition onto a separate applicator. Other application devices may also be suitable including applicators with foam tips, brushes, and the like. Importantly, the applicator must be able to deliver the requisite amount of composition to the tissue. Therefore, in most instances applicator devices such as webs and swabs are coated on the applicator web at greater than 50% by weight of the dry web and preferably in excess of 100% by weight of the dry web. (On a swab this would include the weight only of the web and not the applicator stick.)

The collapsible containers may be made in a number of single layer, laminate, or coextruded constructions. Materials of construction may include polyolefins such as low, medium, or high density polyethylene including low and linear low density polyethylene, polypropylene, as well as copolymers of ethylene and/or propylene with other polar or non-polar comonomers; polyamides such as nylons; polyesters such as polyethylene terephalate, polybutyleneterephalate, polyethylenenaphthalate; polyurethanes; polyacrylates; and the like. In some constructions it may be desirable to include a barrier material to prevent evaporation of one or more components of the formulation. Suitable barrier materials include polyesters (e.g., polyethylene terephthalate, polyethylene naphthalate, polybutylene terephalate, and the like), fluorinated layers such as polytetrafluoroethylene (PTFE, e.g., TEFLON), polyamides (e.g., nylon), chlorotriflouroethylene (ACLAR), polyvinylidene fluoride, as well as copolymers of perflourinated monomers with partially fluorinated monomers such as copolymers of tetraflouroethylene/hexafluoropropylene/vinylidene fluoride (THV Fluorothermoplastic from Dyneon Company), polyvinylchloride, polyvinylidene chloride (PVDC, e.g., SARAN HB), ethylene vinyl alcohol (EVOH), polyolefins (e.g., polyethylene, high density polyethylene, polypropylene, and combinations thereof). Oriented and biaxially oriented polymers may be particularly preferred.

Particularly preferred barrier constructions include metallic foil barriers such as aluminum foil laminates, HDPE, PET, PETG, PEN laminates of polyester and polyolefin (in particular PET/HDPE or HDPE/PET/HDPE), laminates of PET and EVOH, biaxially oriented nylon, PVDC, Nylon/EVOH/Nylon (OXYSHIELD OUB-R), chlorotrifluoroethylene and laminates thereof, ceramic layer including silicon oxide (SiOₓ where x=0.5-2 and preferably 1-2) coated thermoplastics, and ceramic coated PET (CERAMIS available from CCL Container/Tube Division, Oak Ridge, N.J.).

The compositions of the present invention can be delivered from various substrates for delivery to the tissue. For example, the compositions can be delivered from a wipe or pad which when contacted to tissue will deliver at least a portion of the composition to the tissue.

The dose and frequency of application will depend on many factors including the condition to be treated, the concentration of antiviral lipid and enhancer, the microbe to be killed, etc. Typically, the compositions will be delivered in dosages of at least 10 milligrams per square centimeter (mg/cm²) of tissue, preferably at least 20 mg/cm² of tissue, more preferably at least 30 mg/cm of tissue, and most preferably at least 50 mg/cm² of tissue, for most applications. Application can be made once, or several (e.g., 2-6) times daily for one or more days. Typically, the composition is applied 3 to 5 times/day for 1-7 days.

Alternatively (or additionally), the antimicrobial component can include other antimicrobial agents, particularly other antiseptics. Examples of suitable antiseptics include, for example, peroxides, (C6-C14) alkyl carboxylic acids and alkyl ester carboxylic acids, antimicrobial natural oils, as described in Applicants' Assignee's Copending U.S. patent application Ser. No. 10/936,133, filed Sep. 7, 2004; halogenated phenols, diphenyl ethers, bisphenols (including but not limited to p-chloro m-xylenol (PCMX) and triclosan), and halogenated carbanilides described in Applicants' Assignee's Copending U.S. patent application Ser. No. 10/936,171, filed on Sep. 7, 2004; digluconate, diacetate, dimethosulfate, and dilactate salts; polymeric quaternary ammonium compounds such as polyhexamethylenebiguanide; silver and various silver complexes; small molecule quaternary ammonium compounds such as benzalkoium chloride and alkyl substituted derivatives; di-long chain alkyl (C8-C18) quaternary ammonium compounds; cetylpyridinium halides and their derivatives; benzethonium chloride and its alkyl substituted derivatives; and octenidine described in Applicants' Assignee's Copending U.S. patent application Ser. No. 10/936,135, filed on Sep. 7, 2004; and compatible combinations thereof.

Although the detailed description of illustrative embodiments provided herein (particularly with respect to external analgesics, moisturizers, enhancers, other additives, and for making such compositions) specifically refer to an antiviral lipid component, such description also applies to other antimicrobial agents, particularly antiseptics.

### Test Protocols

Herpes Animal Model

Female 23-28 g hairless mice wwere purchased from Charles River Labs (Wilmington, Mass.). They were quarantined one week prior to use, caged in shoebox-style polycarbonate cages with stainless steel tops, and fed standard mouse chow and tap water ad libitum.

Groups of 8 mice each were infected intradermally by lightly scratching the skin on the right shoulder and right hip of the animal using a 20 gauge hypodermic needle using 5 scratches horizontally within a 10 mm diameter square and then placing a drop of 1:10 dilution of the virus on the scratches and rubbing a virus into the scratches with the tip of the pipette.

The virus was a Type 1 herpes virus, strain KOS, initially obtained as a clinical isolate from Dr. Milan Fiala of Harbor General Hospital (Los Angeles, Calif.). It was passaged in Vero cells and titrated in mice prior to use in the experiment.

Topical treatment with all formulations described below began 4 hours after application of the virus, and continuing four times daily (every 6 hours) for 5 days. Treatment was achieved using a Teflon-coated metal spatula, rubbing approximately the same quantity of formulation into each lesion. A standard number of “rubs” was applied to each lesion. The animals were observed daily for the occurrence of death for 21 days.

Each lesion was assigned a score ranging from 0 (normal skin) to 4 (maximal lesion intensity) defined as “Lesion score”, and two measurements, a vertical length and a horizontal length, were taken of each lesion daily from days 1 through 10. These measurements were multiplied together and the “square area” recorded, defined as “Lesion Size”. The lesion scoring was done by technicians who are unaware of which group of animals they are examining in order to eliminate bias. The occurrence of new, satellite, lesions (e.g., another lesion located anywhere other than the site of the intial lesion) were also noted during this 10-day period. The mean of the lesions score and the lesions size was calculated based on the average of the measurements taken on the eight mice.

Two additional mice were used as toxicity controls. The shoulder of each of these animals was scratched as above but not exposed to virus. The formulation was rubbed into both the scratched shoulder and onto intact skin on the hip. These animals were weighed prior to initial treatment and again 18 hours after final treatment. They were also observed daily throughout the treatment for occurrence of skin irritation or other signs of toxicity. Deaths, if they occurred, were recorded daily for 21 days.

### EXAMPLES

Objects and advantages of this invention are further illustrated by the following examples, but the particular materials and amounts thereof recited in these examples, as well as other conditions and details, should not be construed to unduly limit this invention.

### Glossary of Components

| Material | Trade name | Supplier | Address |
|---|---|---|---|
| Propylene | None | Great lakes | St. Paul, MN |
| glycol USP | | Brenntag, LLC | |
| Cetostearyl | Croda | Croda, Inc. | Edision, NJ |
| alcohol NF | cetostearyl | | |
| | alcohol NF | | |
| White | Ultima white | Penereco | Karms City, PA |
| petrolatum | petrolatum | | |
| | USP | | |
| C12-C15 alkyl | Finsolv TN | Finetex, Inc. | Spencer, NC |
| benzoate | | | |
| L-menthol | None | SC | |
| crystals | | Manufacturing | |
| Propylene | Capmul PG-12 | Abitec Corp. | Janesville, WI |
| glycol | | | |
| monolaurate | | | |
| White beeswax | none | Acros | NJ |
| | | chemical | |
| Poloxamer | Pluracare | BASF | Mt. Olive, NJ |
| | P65 | | |
| White | Snow white | Penereco | Karms city, NJ |
| petrolatum | petrolatum | | |
| | USP | | |
| Propyl | Rita propyl | Rita Corp | Woostock, IL |
| paraben | paraben | | |
| Medical grade | Medilan | Croda | Edison, NJ |
| lanolin | Ultra | | |
| Mineral oil | Drakeol 21 | Penereco | Karms City, PA |
| USP | | | |
| Steareth 21 | Brij 721 | Uniquema | Wilmington, De |
| Stearth 2 | Brij 72 | Uniquema | Wilmington, DE |
| Deionized | none | 3M lab, | St. Paul, MN |
| water | | Millipore | |
| | | Unit | |
| Squalane | Phytolane | Barnet | Englewood |
| | Squalane | Products | Cliffs, NJ |
| Olive oil | Bella extra | Lunds | St. Paul, MN |
| | virgin olive | | |
| | oil | | |
| Benzoic acid | none | Mallinkrodt, | St. Louis, MO |
| USP | | Inc. | |
| Tocopherol | Vitamin E | BASF | Mt. Olive, NJ |
| acetate USP | acetate | | |
| carbomer | Ultrez 21 | Noveon | Cleveland, OH |
| Acrylates/ | Pemulen | Noveon | Cleveland, OH |
| C10-30 | TR-2 | | |
| alkyl acrylate | | | |
| crosspolymer | | | |
| Glycerol | Estol 3609 | Uniqema | New Castle, DE |
| tri(2- | | | |
| ethylhexoanote) | | | |
| 50 Centistoke | L-45 | Osi | Wilton, CT |
| polydimethyl | | Specialties | |
| siloxane | | | |
| Glycerin USP | | P&G Chemicals | Cincinnati, OH |
| Ethyl Oleate | none | ISP, Corp. | Somerset, NJ |
| isodocecane | Permethyl | Presperse, | Somerset, NJ |
| | 99A | Inc. | |
| isoeicosane | Permethyl | Presperse, | Somerset, NJ |
| | 102A | Inc. | |
| 1N NaOH | | | |

### Example 1

Mixture A

24.38 g Capmul PG-12

7.21 g cetostearyl alcohol NF

0.22 g propyl paraben

3.95 g white beeswax

4.51 g Brij 721

1.00 g Brij 72

1.01 g squalane

0.51 g L-menthol

Mixture B

4.03 g propylene glycol

3.17 g pluracare P65

0.14 g methyl paraben

50.81 g deionized water

Mixture A in an oil phase was heated to 68° C. in glass vessel on a laboratory hot plate and stirred using a magnetic stir bar for approximately fifteen minutes until the solution became clear. This was then added to a solution of Mixture B at approximately the same temperature of 68° C. under continuous stirring to form a bright white emulsion. The product was cooled to 32° C., whereupon it thickened to form a cream.

### Comparative Example A

Mixture A

9.12 g cetostearyl alcohol NF

2.32 g Brij 721

2.48 g Brij 72

1.33 g squalane

8.11 g Drakeol 21 Mineral oil USP

0.51 g L-menthol

0.21 g propyl paraben

Mixture B

3.46 g propylene glycol USP

1.20 g Pluracare P65

71.41 g deionized water

This formulation was prepared as in example 1. The solution thickened to form a white cream upon cooling.

### Example 2

19.20 g Capmul PG-12

9.95 g white beeswax

1.47 g Pluracare P65

0.34 g L-menthol

0.16 g propyl paraben

1.58 g cetostearyl alcohol NF

11.11 g extra virgin olive oil

6.49 g snow white petrolatum USP

The components were combined in a glass vessel and heated approximately 75° C. until all components were melted. The mixture was stirred and cooled to approximately 50° C. and poured into another glass vessel. Upon cooling to room temperature, the mixture solidified to form a viscous ointment with a pleasant feel.

### Comparative Example B

1.67 g Pluracare P65

3.10 g cetostearyl alcohol NF

5.09 g white beeswax

11.58 g Medilan ultra lanolin

4.11 g Finsolv TN

0.15 g propyl paraben

74.29 g Ultima white petrolatum USP

This formulation was prepared as in example 2. Upon cooling to room temperature, the mixture solidified to form a viscous ointment.

### Example 3

0.259 g benzoic acid USP

0.258 g L-menthol

9.999 g Capmul PG-12

0.642 g tocopherol acetate USP

38.851 g olive oil

All ingredients were combined in a glass vessel and stirred at room temperature (23 deg C.) for 4 hours. The final product formed a light oil which can be directly applied to the skin.

Evaluation of Examples 1-3 in Animal Model

Each of Examples 1-3 and Comparative A-B was tested in the animal model described above using Hairless mice. None of the formulations showed any signs of toxicity on the mice using 2 mice per group for toxicity controls. The results for Examples 1, 2, and 3 along with comparative examples A and B are summarized in Table 1. ZOVIRAX, 5% topical acyclovir ointment (available by prescription at a local pharmacy) was used as a positive control and is also included in Table 1.

TABLE 1

| | | | | Day 7 Mean | Total |
|---|---|---|---|---|---|
| | Surv/ | Mean Day to | Day 7 Mean | Lesion Size | Satellite |
| Example | total | Death ± SD | Lesion Score | (mm²) | Lesions |
| 1 | 6/8 | 15.5 ± 2.1* | 0.5** | 13*** | 0 |
| Compara- | 4/8 | 9.3 ± 1.9 | 1.5 | 82.5 | 7 |
| tive A | | | | | |
| (placebo | | | | | |
| cream) | | | | | |
| 2 | 7/8** | 9.0 ± 0.0 | 0.3*** | 7*** | 0 |
| Compara- | 1/8 | 10.1 ± 2.4 | 2.4 | 168 | 7 |
| tive B | | | | | |
| (placebo | | | | | |
| ointment [symbol] | | | | | |
| 3 | 1/8 | 10.2 ± 2.9 | 1.8 | 107 | 0 |
| 5% | 7/8** | 7.0 ± 0.0 | 0.5** | 40*** | 0 |
| acyclovir | | | | | |

Surv = number of survivors
SD = standard deviation
*P < 0.05;
**P < 0.01;
***P < 0.001 compared to appropriate placebo controls (Comparative A and Comparative B)

Example 1 and Example 2 showed a statistically significant decrease in lesion score and lesion size, comparable to 5% acyclovir, the positive control.

### Example 4

Another mouse study was performed using propylene glycol monolaurate (Capmul PG-12) in a neat composition. The ester was applied 24 hours post-virus inoculum 3 times daily for 5 days. The neat liquid ester showed a statistically significant reduction in lesion score and size compared to a placebo ointment. The mean day 7 lesion size was 3.1 mm2 compared to 64.4 mm2 for petrolatum vehicle control. The day 7 mean lesion score was 0.2 compared to 1.5 for a control sample containing petrolatum without an antimicrobial.

### Example 5

An ointment was prepared by charging 19.5 grams propylene glycol monolaurate, 2.5 grams ethyl oleate, 3 grams glycerol tri(2-ethylhexanoate), 0.75 g tocopheryl acetate, 0.4 grams menthol, 0.15 grams propyl paraben, 1 gram of 50 centistokes polydimethylsiloxane fluid, 10 grams of beeswax, 6.25 grams of isododecane, 6.25 grams of isoeicosane, and 0.2 grams of butter rum flavoring agent to a 4 ounce glass jar. This was immersed in a water bath held at 65 C, and was mixed with an overhead stirrer and a propeller blade at a moderate rate for 30 minutes at which point the solid materials had dissolved and the waxes had melted giving a clear yellow solution. The jar was removed from the water bath and allowed to cool overnight at room temperature yielding a hazy ointment.

### Example 6

An oil-in-water cream was prepared by charging 12.5 grams propylene glycol monolaurate, 1.25 grams ethyl oleate, 0.75 g tocopheryl acetate, 0.5 grams Pluracare P65 polaxomer, 0.4 grams menthol, 0.08 grams propyl paraben, and 0.05 grams methyl paraben to a 4 ounce glass jar. This was mixed at room temperature with an overhead stirrer and a propeller blade at a moderate rate and 0.5 grams allantoin, 0.12 grams Pemulen TR-2, and 0.05 grams Ultrez 21 were added. To the resulting suspension of powders in oil was added a solution of 2 grams 1N aqueous NaOH, 2.5 grams of glycerine, and 29.3 grams water. The stirring rate was increased to ensure good mixing of the now viscous white cream while at the same time not so high as to entrain air via vortexing. After 30 minutes of stirring at room temperature a creamy white emulsion that holds peaks resulted. The measured pH was 7.6.

Evaluation of Examples 5 and 6 in Animal Model

Each of Examples 5 and 6 as well as Example 2 (as a retest) was tested in the animal model described above using Hairless mice. None of the formulations showed any signs of toxicity on the mice using 2 mice per group for toxicity controls. The results for Examples 2, 5, and 6 along with an untreated control are summarized in Table 1. ZOVIROX, 5% topical acyclovir ointment (available by prescription at a local pharmacy) was used as a positive control and is also included in Table 1. Other over-the-counter cold sore medications including Neosporin LT Lip Treatment and Abreva were investigate in this study as well. Abreva is an over-the-counter drug approved by the FDA for treating cold sores.

TABLE 2

| | | | | Day 7 Mean | Total |
|---|---|---|---|---|---|
| | Surv/ | Mean Day to | Day 7 Mean | Lesion Size | Satellite |
| Example | total | Death ± SD | Lesion Score | (mm²) | Lesions |
| 2 | 8/8 | >21 ± 0.0*** | 0.3*** | 3.1*** | 1*** |
| 5 | 4/8 | 11.5 ± 21 | 0.8 | 28.3 | 6 |
| 6 | 7/8** | 11.0 ± 0.0 | 0.2*** | 2.6*** | 0*** |
| Neosporin | 1/8 | 9.3 ± 2.2 | 2.3 | 149.1 | 24 |
| LT | | | | | |
| Abreva | 1/8 | 10.3 ± 1.5 | 1.8 | 106.5 | 18 |
| 5% | 7/8** | >21 ± 0.0*** | 0.0*** | 0.0*** | 0*** |
| acyclovir | | | | | |
| Untreated | 5/8 | 10.3 ± 1.5 | 1.3 | 54.4 | 17 |

Surv = number of survivors
SD = standard deviation
*P < 0.05;
**P < 0.01;
***P < 0.001 compared to untreated

As shown in Table 2, the compositions of the present invention are useful for treating viral infections such as Herpes Simplex I.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of treating a viral infection caused by the herpes virus in or on the skin or mucuous membranes of a subject, the method comprising contacting the affected area with an antiviral composition comprising:
an effective amount of an antiviral lipid component comprising a (C7-C12) saturated fatty acid ester of a polyhydric alcohol, a (C8-C22) unsaturated fatty acid ester of a polyhydric alcohol, a (C7-C12) saturated fatty ether of a polyhydric alcohol, a (C8-C22) unsaturated fatty ether of a polyhydric alcohol, a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, wherein the alkoxylated derivative has less than 5 moles of alkoxide per mole of polyhydric alcohol; and
an external analgesic.

2. The method of claim 1 wherein the antiviral lipid component is present in an amount greater that 5 wt-%.

3. The method of claim 1 wherein the antiviral lipid component is present in an amount greater that 15 wt-%.

4. The method of claim 1, further comprising a moisturizer.

5. The method of claim 4, wherein the moisturizer comprises a humectant, an emollient, and combinations thereof.

6. The method of claim 5 wherein the humectant comprises a glycol, urea, glycerol, and combinations thereof.

7. The method of claim 1, further comprising a hydrophobic component separate from the antiviral lipid component.

8. The method of claim 1 wherein the antiviral lipid component further comprises no greater than 15 wt-%, based on the total weight of the antiviral lipid component, of a di- or tri-ester, a di- or tri-ether, alkoxylated derivative thereof, or combinations thereof.

9. The method of claim 1, wherein the external analgesic is selected from the group consisting of benzocaine, butamben picrate, dibucaine, dibucaine HCl, dimethisoquin HCl, dyclonine HCl, lidocaine, lidocaine HCl, pramoxine HCl, tetracaine, tetracaine HCl, benzyl alcohol, camphor, camphorated metacresol, juniper tar, menthol, phenol, phenolate sodium, resorcinol, diphenhydramine HCl, tripelennamine HCl, hydrocortisone, hydrocortisone acetate, and mixtures thereof.

10. The method of claim 1, further comprising a skin protectant.

11. The method of claim 10, wherein the skin protectant is selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

12. The method of claim 1 wherein the antiviral lipid component comprises an effective amount of an antiviral lipid component comprising a (C7-C14) saturated fatty acid ester of propylene glycol, a (C8-C22) unsaturated fatty acid ester of propylene glycol; and combinations thereof

13. The method of claim 1 wherein the antiviral lipid component comprises propylene glycol monolaurate, propylene glycol monocaprate, propylene glycol monocaprylate, or combinations thereof.

14. The method of claim 1 further comprising a surfactant.

15. The method of claim 14 wherein the surfactant is a nonionic surfactant.

16. A method of killing or inactivating microorganisms, the method comprising contacting the microorganisms with an antiviral composition comprising propylene glycol fatty acid monoester in an amount greater than 20 wt %.

17. The method of claim 16 wherein the microorganisms comprise one or more viruses and the antiviral composition is used in an amount effective to inactivate one or more viruses.

18. A method of treating and/or preventing a viral infection on mammalian tissue of a subject, the method comprising contacting the mammalian tissue with an antiviral composition in an amount effective to kill or inactivate one or more microorganisms, wherein the antiviral composition comprises:
an effective amount of an antiviral lipid component comprising a (C7-C12) saturated fatty acid ester of a polyhydric alcohol, a (C8-C22) unsaturated fatty acid ester of a polyhydric alcohol, a (C7-C12) saturated fatty ether of a polyhydric alcohol, a (C8-C22) unsaturated fatty ether of a polyhydric alcohol, a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, wherein the alkoxylated derivative has less than 5 moles of alkoxide per mole of polyhydric alcohol; and
an external analgesic.

19. A topical antiviral composition comprising:
an antiviral lipid component comprising a (C7-C14) saturated fatty acid monoester of a polyhydric alcohol, a (C8-C22) unsaturated fatty acid monoester of a polyhydric alcohol, a (C7-C12) saturated fatty monoether of a polyhydric alcohol, a (C8-C22) unsaturated fatty monoether of a polyhydric alcohol, a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, present in an amount greater than 5% based on the total weight of the composition; and
an external analgesic.

20. The composition of claim 19 wherein the antiviral lipid is present in an amount greater that 10 wt-%.

21. The composition of claim 19 wherein the antiviral lipid is present in an amount greater that 15 wt-%.

22. The composition of claim 19, further comprising a moisturizer.

23. The composition of claim 22, wherein the moisturizer comprises a humectant, an emollient, and combinations thereof.

24. The composition of claim 23 wherein the humectant comprises a glycol, ureas, glycerol, and combinations thereof.

25. The composition of claim 19, wherein the external analgesic is selected from the group consisting of benzocaine, butamben picrate, dibucaine, dibucaine HCl, dimethisoquin HCl, dyclonine HCl, lidocaine, lidocaine HCl, pramoxine HCl, tetracaine, tetracaine HCl, benzyl alcohol, camphor, camphorated metacresol, juniper tar, menthol, phenol, phenolate sodium, resorcinol, diphenhydramine HCl, tripelennamine HCl, hydrocortisone, hydrocortisone acetate, and mixtures thereof.

26. The composition of claim 19, further comprising a skin protectant.

27. The composition of claim 26, wherein the skin protectant is selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

28. The composition of claim 19 wherein the antiviral lipid component comprises propylene glycol monolaurate, propylene glycol monocaprate, propylene glycol monocaprylate, or combinations thereof.

29. A method of treating herpes lesions on or in the skin or mucuous membranes of a subject, the method comprising contacting the affected area with an antiviral composition comprising:
an effective amount of an antiviral lipid component comprising a (C7-C12) saturated fatty acid ester of a polyhydric alcohol, a (C8-C22) unsaturated fatty acid ester of a polyhydric alcohol, a (C7-C12) saturated fatty ether of a polyhydric alcohol, a (C8-C22) unsaturated fatty ether of a polyhydric alcohol, a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, wherein the alkoxylated derivative has less than 5 moles of alkoxide per mole of polyhydric alcohol; and
an external analgesic.

30. The method of claim 29 wherein in the herpes lesion is present on mucosal tissue.

31. A method of treating viral infection on or in the skin or mucuous membranes of a subject, the method comprising contacting the affected area with an antiviral composition comprising:
an effective amount of an antiviral lipid component comprising a (C7-C14) saturated fatty alcohol monoester of a (C2-C8)hydroxycarboxylic acid, a (C8-C22) mono- or poly-unsaturated fatty alcohol monoester of a (C2-C8)hydroxycarboxylic acid, an alkoxylated derivative thereof, or combinations thereof, wherein the alkoxylated derivative has less than 5 moles of alkoxide per mole of polyhydric alcohol.
